(19) United States
(12) Patent Application Publication
(10) Pub. No.: US 2018/0085290 A1
(43) Pub. Date: Mar. 29, 2018

(54) METHOD AND DEVICE FOR DEPOSIT PARTICLE LAYERS USING ALTERNATING DELIVERY OF POSITIVELY AND NEGATIVELY CHARGED PARTICLES

(71) Applicant: KONINKLIJKE PHILIPS N.V., EINDHOVEN (NL)

(72) Inventor: BART GOTTENBOS, BUDEL (NL)

(21) Appl. No.: 15/562,931

(22) PCT Filed: Apr. 1, 2016
(86) PCT No.: PCT/IB2016/051869

§ 371 Date: Sep. 29, 2017

Related U.S. Application Data
(60) Provisional application No. 62/141,613, filed on Apr. 1, 2015.

(51) Int. Cl. A61K 8/02; A61Q 11/00

(52) U.S. Cl. CPC A61K 8/0266; A61K 2800/92; A61K 2800/87; A61Q 11/00

(57) ABSTRACT

A delivery system includes a first layer which includes at least one of positively-charged particles and positively-charged molecules. A second layer is in contact with the first layer. The second layer includes at least one of negatively-charged particles and negatively-charged molecules. At least one of the first and second layers includes an active agent, such as an oral care agent. The active agent is incorporated in the positively-charged particles and/or negatively-charged particles of the respective layer.

### BACKGROUND

The following relates to the dental care arts, and related arts and more specifically concerns a system and method for applying an oral care agent.

Oral care agents, such as antiplaque agents, anti-tartar agents, anti-gingivitis agents, anti-bacterial agents, and tooth whitening agents are often administered to the human oral cavity, in particular, to the teeth and gums, from toothpastes and oral rinse liquids. Oral care agents in toothpastes and oral rinses tend to reduce quickly in concentration after their application due to the presence of saliva in the mouth. They are thus unable to provide long-term protection to the teeth or gums (e.g., up to 24 h).

Coating the teeth or mucosa with sustained release particles that can contain and slowly release oral care agents is one approach for maintaining the concentration of oral care agents for longer times. In addition to being able to release the oral care agent at a suitable rate, it is also desirable for the particles to adhere to oral tissue in order to be delivered effectively. As oral tissue surfaces are typically negatively charged, using particles with a net positive surface charge can facilitate a fast and long lasting adhesion of the particles to the oral surfaces.

Though positively charged particles adhere very well to the oral surfaces, they tend to repel each other. This has an advantage in that discrete particles are maintained in suspension prior to delivery, rather than agglomerating. However, a disadvantage is that only about a monolayer of the particles can be deposited on the oral surface. As particles are often below a size of 0.2 mm to avoid clogging deposition devices, this limits the thickness of the layer of slow release particles that can be deposited, and thus the amount of oral care agent that is available for sustained release. For a desired efficacy of any oral care agent there is generally a minimum effective concentration, and for safety there is often also a maximum permitted concentration. This often limits the concentration of oral care agent in each particle that is deposited.

For many oral care agents, therefore, it would be advantageous to be able to apply more than a monolayer of particles to the surface in order to provide a minimum effective concentration and/or an optimum amount of the oral care agent.

A system and method are disclosed which can overcome some of the problems with existing systems.

One advantage of the exemplary system is that a volume of particles delivered to a surface in the oral cavity is not limited to a monolayer.

### BRIEF DESCRIPTION

In accordance with one aspect of the invention, a delivery system includes a first layer which includes at least one of positively-charged particles and positively-charged molecules. A second layer is in contact with the first layer. The second layer includes at least one of negatively-charged particles and negatively-charged molecules. At least one of the first and second layers includes an active agent. The active agent is incorporated in the positively-charged particles or negatively-charged particles of the respective layer.

In accordance with another aspect of the invention, a deposition method includes depositing positively-charged particles on a surface of an oral cavity, depositing at least one of negatively-charged particles and negatively-charged molecules on the positively-charged particles. An oral care agent is incorporated in at least one of the positively-charged particles and the negatively-charged particles, where present. Optionally, the depositing of positively-charged particles on the at least one of negatively-charged particles and negatively-charged molecules is repeated.

In accordance with another aspect of the invention, a deposition device includes a first reservoir which holds positively-charged particles, a second reservoir which holds negatively-charged particles, at least one of the positively-charged particles and negatively-charged particles incorporating an active agent. A delivery mechanism delivers the positively charged particles and the negatively-charged particles to a surface.

### BRIEF DESCRIPTION OF THE DRAWINGS

The invention may take form in various components and arrangements of components, and in various process operations and arrangements of process operations. The drawings are only for the purpose of illustrating preferred embodiments and are not to be construed as limiting the invention.

FIG. 1 diagrammatically shows a delivery system for applying an oral care agent in accordance with one embodiment disclosed herein.

FIG. 2 schematically illustrates three layers of particles.

FIG. 3 is a flow chart illustrating a method for forming the exemplary delivery system, in accordance with embodiments disclosed herein.

FIG. 4 illustrates a deposition device for applying the exemplary delivery system in accordance with another embodiment disclosed herein, during application of a first layer of the delivery system.

FIG. 5 illustrates the deposition device of FIG. 4, during application of a second layer of the delivery system.

FIG. 6 illustrates the deposition device of FIG. 4, during application of a third layer of the delivery system.

FIG. 7 illustrates another embodiment of a deposition device.

FIG. 8 shows macroscope images of particle deposits (chitosan particles white, alginate transparent) on black tooth shaped surfaces, for a control group having only chitosan particles deposited.

FIG. 9 shows macroscope images of particle deposits (chitosan particles white, alginate transparent) on black tooth shaped surfaces for a test group alternating chitosan and alginate particles.

### DETAILED DESCRIPTION

Exemplary embodiments relate to a deposition device, a method, and a delivery system for supplying an active agent, such as an oral care agent to a surface of a person or an animal, such as to the oral cavity, e.g., to the teeth and/or gums.

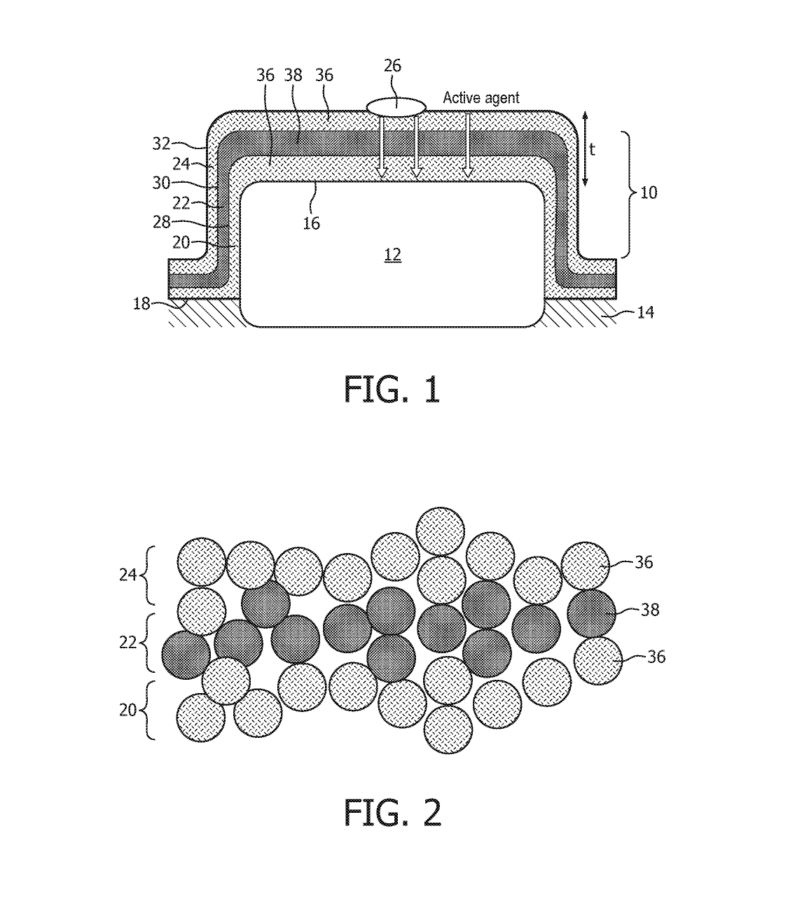

With reference to FIG. 1 (not to scale), a schematic side sectional view of multilayer delivery system 10 for delivery of an oral care agent to a surface of an oral cavity is shown. FIG. 1 illustrates only a portion of an oral cavity of a human or animal, including a tooth 12 and associated portion of the gums 14. The multilayer system 10 may cover at least a portion of a surface 16, 18 of the teeth and/or gums of the wearer. The multilayer system 10 includes a plurality of layers 20, 22, 24, such as two, three, four, or more layers, such as up to a hundred layers, although three are shown for ease of illustration. The number of layers employed may depend on the size of the particles, the desired concentration of the oral care agent in the oral cavity/on the teeth or gums, the desired release time, and/or other factors. Some or all of the layers 20, 22, 24 include an oral care agent, which may be the same or different for each layer. In the presence of saliva (water) 26, oral care agent flows to the surface 16, 18 from the particles.

As illustrated in FIG. 2, each of the layers 20, 22, 24 of the delivery system includes particles which incorporate an oral care agent. Each layer is typically a monolayer (single layer of particles) in thickness and is in contact with particles of opposite polarity. The second layer 22 of particles spaces the first layer 20 from the third layer 24, at least in part. In particular, an outer surface 28 of the first layer contacts the second layer 22 and an outer surface 30 of the second layer 22 is in contact with the third layer, and so forth, with the outer surface 32 of the outermost layer 24 being exposed to the mouth cavity. The particles of the layers can carry a charge. In particular, particles 36 of the first layer 20, which are in contact with the tooth or gum surface 16, 18 carry a positive charge, particles 38 of the second layer 22, intermediate and in contact with the first and third layers, carry a negative charge, and particles 36 (or different particles) of the third layer 24 carry a positive charge (and may be similarly constituted and be formed in the same way as for the particles 36 of the first layer). In this way, the charge of the layers alternates. The outermost layer particles may carry a positive or negative charge, depending on the number of layers.

As will be appreciated, the layers may not be discrete, as shown in FIGS. 1 and 2, but rather the alternating application of positively and negative-charged particles to the teeth and/or gums results in a loose mixture of particles in which positively-charged articles are interspersed with negatively-charged particles.

In some embodiments, over time, the outer layer 24 is eroded away revealing the next layer 22, and so forth, e.g., over the course of a few hours or some other time period.

An average thickness t of the resulting system 10 may be up to 5 mm, and can be at least 0.1 mm.

The particles can be solid particles, gel particles, vesicles, or other three-dimensional structures. The particles serve to provide a controlled release, e.g., a sustained release, of oral care agents therefrom. This enables the concentration of oral care agents to be maintained at a minimum level for longer times or reduces the decrease in concentration of oral care agents.

The particles may be at least 10 μm in size, such as at least 20 μm in size, or at least 50 μm, or at least 100 μm. The particles may be up to 0.2 mm in size, such as up to 100 μm. By size, it is meant the average (mean) particle size, as determined by microscope imaging of particles in suspension when buffered to about neutral pH (pH 6.5-pH 7.5), with phosphate buffer, on a black background.

In one embodiment, the particles are gel particles. Gels generally have a low volume of solids (e.g., 1-2%) and can therefore contain a large volume of oral care agents.

The positively-charged particles have net positive surface charge and the negatively-charged particles have a net negative surface charge. This means that the positively-charged particles may have a zeta potential of greater than 0 and the negatively-charged particles have a zeta potential of less than 0 in neutral media (pH 7.0). Zeta potential ζ is obtained from the Helmholtz-Smoluchowski equation:

$$ζ = \frac{4 πη}{ɛ} f ( κα ) \cdot {μ}_{e}$$

where ∈=Dielectric constant
η=Viscosity of medium
f(κa)=Debye function, which may be approximated to 1.0
μₑ=Electrophoretic mobility

$$( {μ}_{e} = \frac{v}{E} )$$

ν=Velocity of particle in E-field
E=Electrical field

Electrophoretic mobility can be obtained by subjecting samples of the particles in a selected medium to electrophoresis and measuring the velocity of the particles, e.g., using particle image velocimetry (PIV) or laser Doppler velocimetry (LDV). Particle image velocimetry (PIV) is used in the exemplary embodiment as it is more suitable to measuring the velocity of larger particles.

In general the zeta potential of the positively-charged particles is at least 1 mV, or at least 5 mV, or at least 10 mV, and the zeta potential of the negatively-charged particles is at least as low as −1 mV, at least as low as −5 mV, or at least as low as −10 mV, or at least as low as −30 mV, in neutral media. A difference in zeta potential between the positively-charged particles and the negatively-charged particles may be at least 2 mV, or at least 2 mV, or at least 10 mV, or at least 15 mV, or at least 20 mV.

The hydrogel particles include a gel matrix derived from a gel-forming, biocompatible polymer which, in turn, is derived from one or more polymerizable monomers or directly derived from nature (such as chitosan, from chitin, and alginate). Biocompatible polymers, as defined herein are those which, together with any degradation products of the polymer, are non-toxic to the recipient and also present no significant deleterious or untoward effects on the recipient's body. Suitable polymers include polysaccharides, such as chitosan, alginate and cellulose, poly-lactic acid (PLA), poly-glycolic acid (PGA), poly-lactide-co-glycolide (PLGA), polyesters, poly(ortho ester), poly(phosphazine), poly(phosphate ester), polycaprolactone, gelatin, collagen, fibronectin, keratin, polyaspartic acid, chitin, hyaluronic acid, pectin, polyhydroxyalkanoates, dextrans, and polyanhydrides, polyethylene oxide (PEO), poly(ethylene glycol) (PEG), polylysine, and copolymers, derivatives, and mixtures thereof.

Desirably, the positively-charged gel is a mucoadhesive gel. Suitable positively-charged gels (at neutral pH) include:
those derived from basic polysaccharides, such as chitosan and modified derivatives thereof, which are water soluble, non-toxic, biocompatible and biodegradable.
(meth)acrylate and vinyl polymers: cross linked acrylic acid-based polymers present swellable behaviour in aqueous solutions due to the presence of ionizable functional groups. Under certain pH they acquire charge and the electrostatic repulsion between these groups favours the intake of water and the expulsion of the agent. This feature makes them suitable candidates for pH-triggered controlled release, at specific sites. Examples of these polymers include carbomers sold under the tradename Carbopol®. Positively charged functional groups, such as amines and quaternary ammonium groups, can be used to make such polymers more positive.
polysaccharides, such as chitosan, in combination with poly (acrylic acid) and/or poly (methyl methacrylate), can be used to produce cross linked micro and nanoparticles for controlled release of proteins, vaccines, pharmaceutical compounds and pesticides.
Poly (β-amino ester) polymers can be used to design pH-responsive polymer microspheres. Such systems degrade slowly at pH 7.4 but enable a fast and quantitative release (up to 90% of the encapsulated agent) in acidic conditions, which is useful in biomedical applications to achieve specific different release rates within the physiological pH of the specific site.

Other suitable positively-charged gels are described, e.g., in Fini et al., Pharmaceutics 3, 665-679 (2011).

Chitosan is a linear polysaccharide composed of randomly distributed β-(1-4)-linked D-glucosamine and N-acetyl-D-glucosamine and is generally derived by deacetylation of chitin, which is present in the shells of crustaceans and some fungi. As chitosan is readily available as a food grade material, which can create strong hydrogels, it is particularly useful for forming oral gel particles.

Suitable negatively-charged gels (at neutral pH) include those derived from acidic polysaccharides, such alginate, poly-glycolic acid (PGA), poly-lactide-co-glycolide (PLGA); poly(phosphate ester); polyaspartic acid; hyaluronic acid. Mildly negatively charged at neutral pH: cellulose; dextrans; polyethylene oxide (PEO); poly(ethylene glycol) (PEG), and modified derivatives thereof. Alginates, for example, are salts of alginic acid, such as alkali metal salts (e.g., sodium, calcium, or magnesium salts) or organic salts, such as alginate esters, e.g., propylene glycol alginate, and mixtures thereof.

Lactic and glycolic acid-based polymers show excellent biocompatibility and hydrophilic nature, which makes them good choices for controlled release and drug delivery.

Cellulose-derived polymers, which present different hydrophilicity, swelling and degradation behavior, offer a flexible and tuneable mechanism for controlled release. Commercial examples of these materials include ETHOCEL™, METHOCEL™ and POLYOX™. They can be used to form negatively-charged particles. Mixed inorganic-organic polymers, such as silicones, can also be used to form negatively-charged particles.

Acidic groups such as carboxylic acid groups, sulfate groups and phosphate groups can be used to give gel particles a negative zeta potential at neutral pH. An exemplary sulfated polymer is carrageenan, which can form a gel with divalent cations (e.g., calcium), similar to alginate. (Meth)acrylate polymers can be functionalized with phosphate, sulfate, and/or acid groups to give them a negative zeta potential at neutral pH.

Polysaccharides have several reactive groups that are available for chemical modification. These include the hydroxyl (OH), carboxyl (COOH), and acetamido (COCH₃) groups. Further functionality can be imparted to specific polysaccharides in the form of an amine (NH₂) group via basic deacetylation, in which a polysaccharide is exposed to basic conditions at elevated temperatures. The degree of deacetylation is dependent on the strength of the alkaline conditions, the temperature of the reaction environment, and the duration of the reaction. For example, the percentage of deacetylation can be controlled to obtain different chitosan molecules from a single source of chitin. Other methods of imparting functionality onto polysaccharides include the functionalizing of native hyaluronic acid with amine groups through the use of a hydrazide (see, e.g., U.S. Pat. No. 5,874,417). In this method, the carboxyl group of the disaccharide is linked to a multi-functional hydrazide under acidic conditions in the presence of a soluble carbodiimide.

Mixtures of polysaccharides with other polymers can be used, such as a blend of a basic polysaccharide such as chitosan and anionic polysaccharide such as hyaluronic acid; a blend of alginate and oxidized alginate with chitosan; a grafted agar and sodium alginate blend with acrylamide; gellan co-crosslinked with scleroglucan; photocrosslinked modified dextran; starch reacted with glycidyl methacrylate; and polymerizable saccharide monomers, such as sucrose, created by reaction of the sugar with epoxy acrylate, or methacryloyl chloride and acetyl chloride.

The particles 36, 38 can be incorporated into a mouthwash, toothpaste, or other oral care product.

### Oral Care Agents

One or more of the exemplary particles 36, 38 may include an oral care agent. The oral care agent can include a tooth whitening agent, such as a bleaching agent, and/or other dental care agents, such as a fluoride (e.g., NaF), an antibacterial agent, a remineralization agent, an anti-plaque agent, a pain relief agent (e.g., KNO₃), an anti-odor agent, a long-term protective component, a reactive enzyme, a reactive radical, a combination thereof, or the like.

For improving the efficacy of oral therapeutic agents such as fluoride and/or antimicrobial agents, the use of sustained or controlled release particles is a very attractive solution.

Particular Examples of these Agents Include:

Whitening agents: The oral care agent may be/include a whitening (e.g., bleaching) agent. Example bleaching agents include hydrogen peroxide, carbamide peroxide and other hydrogen peroxide complexes, alkali metal percarbonates, perborates, such as sodium perborate, persulfates, such as potassium persulfate, calcium peroxide, zinc peroxide, magnesium peroxide, strontium peroxide, peroxyacids, sodium chlorite, combinations thereof, and the like. The term “bleaching agent,” herein refers to compounds which are themselves bleaches, such as hydrogen peroxide, and to compounds which are bleach precursors, such as carbamide peroxide, which react or decompose to form a bleach, such as hydrogen peroxide.

Tartar control (anticalculus) agents: these may include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-dipho sphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts, and mixtures thereof.

Fluoride ion sources: These may be useful, for example, as an anti-caries agent. Orally acceptable fluoride ion source which can be used include potassium, sodium and ammonium fluorides and monofluorophosphates, stannous fluoride, indium fluoride and mixtures thereof.

Tooth and soft tissue desensitizers: these may include stannous ions, such as halides and carboxylate salts, arginine, potassium citrate, potassium chloride, potassium tartrate, potassium bicarbonate, potassium oxalate, potassium nitrate, strontium salts, and mixtures thereof.

Antimicrobial (e.g., antibacterial) agents: these may include orally acceptable antimicrobial agents, such as Triclosan (5-chloro-2-(2,4-dichlorophenoxy)phenol); 8-hydroxyquinoline and salts thereof, zinc and stannous ion sources such as zinc citrate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride); bisguanides, such as chlorhexidine digluconate; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; grapeseed extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; and mixtures thereof. Other useful antimicrobials are disclosed in U.S. Pat. No. 5,776,435.

Antioxidants: orally acceptable antioxidants which can be used include butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Antiplaque (e.g., plaque disrupting) agent: orally acceptable antiplaque agents can include stannous, copper, magnesium and strontium salts, dimethicone copolyols such as cetyl dimethicone copolyol, papain, enzymes, such as glucoamylase, glucose oxidase, dextranase, DNase, RNase, lipase, protease, and bromelain, urea, calcium lactate, calcium glycerophosphate, strontium polyacrylates, and mixtures thereof.

Anti-caries agents: examples of these include amorphous calcium phosphate (ACP), calcium glycerylphosphate and sodium trimetaphosphate.

Anti-inflammatory agents: orally acceptable anti-inflammatory agents can include steroidal agents, such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone, and mixtures thereof.

H₂ antagonists: antagonists useful herein include cimetidine, etintidine, ranitidine, ICIA-5165, tiotidine, ORF-17578, lupititidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408.4, and mixtures thereof.

Nutrients: Suitable nutrients include vitamins, minerals, amino acids, proteins, and mixtures thereof.

The concentration of the oral care agent in the particles may depend on the desired concentration at the tooth or gum and/or its delivery rate. Since the system can be relatively thick, the concentration can be lower than for particles in a monolayer and yet deliver the same oral care benefit. Hydrogels tend to be low in solids e.g., 0.5-5% solids, such as 1% or 2% solids, so they are able to hold a significant quantity of the oral care agent. As an example, the oral care agent (expressed as the amount of actives) may be at least 0.001 wt. % of the weight of the particles, and can be up to 20 wt % or up to 10% of the weight of the particles.

For example, antimicrobials may be present in weights comparable to those found in mouthwashes, such as in amounts ranging from 0.01 to 5% by weight with respect to the total weight of the particles. These oral care agents may also serve as plaque-reducing agents.

Oral care agents typically found in mouth washes and toothpastes may be used at similar concentrations, by weight with respect to the total weight of the particles, expressed as the amount of actives. For example, cetyl pyridinum chloride (CPC) may be used in concentrations up to 0.1 wt. %. It may be combined with a zinc compound at a total concentration of at least 0.01 wt. %, for example in the form of zinc chloride, gluconate and/or citrate at a concentration of up to 3 wt. %, or at least 0.1 wt %. Zinc gluconate may be used at about 0.75 wt. %, while zinc citrate may be used at about 1 wt. %, for example. Stannous fluoride may be present at up to 1 wt. %, such as at least 0.1 wt. %. Chlorhexidine digluconate may be used at up to 0.5 wt. %, or at least 0.1 wt. %. Triclosan may be used in concentrations of up to 1 wt. %, or at least 0.05 wt %. Fluorides, such as sodium fluoride, may be present at up to 5 wt. %, or at least 0.5 wt. % NaF. Essential oils, such as eucalyptol, menthol, thymol, and methyl salicylate, may be present at a total concentration of up to 2 wt. %, or at least 0.05 wt.

In some embodiments, the oral care agent is present in the positively-charged particles but not in the negatively-charged particles, or is only present in the negatively-charged particles in a much lower concentration than in the positively-charged particles (such as up to 20% by weight of the concentration in the positively-charged particles, or at least 1 wt. %).

In some embodiments, the oral care agent is present in the negatively-charged particles but not in the positively-charged particles, or is only present in the positively-charged particles in a much lower concentration than in the negatively-charged particles (such as up to 20% by weight of the concentration in the negatively-charged particles, or at least 1 wt. %).

In some embodiments, the positively-charged particles include a first oral care agent and the negatively-charged particles may include a second (different) oral care agent. The first oral care agent is not present in the negatively-charged particles, or is only present in the negatively-charged particles in a much lower concentration (as described above). The second oral care agent is not present in the positively-charged particles or is only present in them in a much lower concentration (as described above).

These embodiments are suited to cases where the oral care agent is retained in the positively- and negatively-charged particles differently, such that the release rate differs. For example, the release rate from one type of particle may be too fast or too slow for effective oral care treatment. In other embodiments, a difference in release rate from the two types of particles is exploited to give an extended release of a single oral care agent-by having one type of particles provide an initial quick release of the oral care agent and the other type a slow release of the same oral care agent, so as to maintain a desired concentration for an extended period.

In some embodiments, it may desirable to have the first oral care agent release slowly and the second (different) oral care agent release more quickly.

In some embodiments, the first oral care agent may be incompatible with the second oral care agent, making it desirable to keep them separated until applied in the oral cavity. For example, hydrogen peroxide may be kept separate from an accelerating agent which enhances the activity of the hydrogen peroxide. In another embodiment, two reagents may be kept separate which combine to form the oral care agent in situ when mixed in the mouth.

### Delivery of Oral Care Agents to the Oral Cavity

With some types of particles, such as chitosan hydrogel particles, there may be little or no erosion or dissolution of the gel component of the particles, at least for a few hours or up to 24 hours or more if the mechanical and shear forces remain low enough, such as between the teeth. In this embodiment, the oral care agents may be released by desorption from the polymer matrix and subsequent diffuse out of the particles. Good sustained release of charged active agents, such as cetyl pyridinium ions or zinc ions (both positively charged) can be achieved in this way. This is likely because they can only slowly desorb from the negative charges available in the gel matrix. Even though the chitosan gel has a net positive surface charge, the matrix still contains many negatively charged groups which can bind positively charged active agents. Similarly negatively charged agents (e.g., fluoride) can be bound by the positively charged groups.

As will be appreciated, the oral care agents can also be transported by diffusion from the inner layers to the outer layers, depending on the particle properties. For example, the agents can diffuse away in all directions, i.e., not only to the tooth surface, but towards the outer layer, in this way, the concentration of the oral care agents can be high enough to, for example, inhibit growth of dental plaque.

The oral care agent(s) may release from the system 10 over a period of at least 2 hours and in some embodiments, up to 24 hours or longer. In one embodiment, at least 10 wt. %, of the original weight of the oral care agent in the system 10, and which is ultimately released, remains in the system after at least 2 hours.

### Method of Forming the Delivery System:

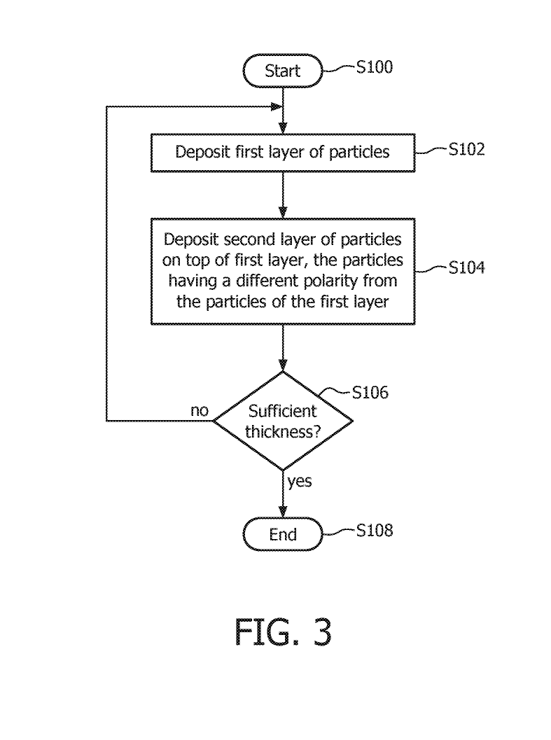

With reference to FIG. 3, a method for forming the multilayer delivery system 10 is illustrated. The method begins at S100. The surface 16, 18 of the oral cavity on which the system is to be applied may be treated, for example by drying the surface, prior to application of the first layer. At S102, a first layer is deposited on the tooth surface, such as a layer 20 containing positively-charged particles 36, which may be deposited in a first suspension fluid, such as a gas e.g., air, or a liquid, e.g., water, aqueous solution, organic liquid, or combination thereof. In some embodiments, the fluid is at or near neutral pH (pH 6.5-7.5). In other embodiments, the pH may be higher or lower. For example the particles may be formulated at a pH as low as 4. Below pH 4 may be less safe for the oral cavity (possible erosion of teeth). After deposition, a period of time or an active drying method may be employed to remove some of the liquid, if the suspension fluid contains a liquid.

At S104, a second layer is deposited on top of the first layer, such as a layer 22 containing negatively-charged particles 38, which may be deposited in a second suspension fluid, such as a gas e.g., air or a liquid, e.g., water, aqueous solution, organic liquid, or combination thereof (which may be the same as the first suspension fluid). After deposition, a period of time or an active drying method may be employed to remove some of the liquid, if the suspension fluid contains a liquid.

At S106, if more layers are desired, the method returns to S102, when a third layer is deposited on top of the second layer, such as a layer 24 containing positively-charged particles 36, and so on until the desired number of layers and/or thickness is achieved. The method ends at S108.

Alternating deposition of particle suspensions, starting with the positively charged particles, as the oral surfaces are negatively charged, results in multiple layers of particle being deposited, leaving a large volume of conglomerated particles on the oral surfaces. It should be noted that while using two particle suspensions renders the largest volume deposited, with the least amount of deposition shots, it is also contemplated that one of the particle suspensions could be replaced with a solution of positively or negatively charged molecules that can form an oppositely charged film on the particle suspension fluid. For example a positively-charged particle suspension could be alternated with a negatively-charged polymer solution.

### Deposition Device

In one embodiment, the method of FIG. 3 may be implemented simply by alternately rinsing with each of the particle suspensions, and then spitting out the rinse before rinsing with the next rinse. However, this is generally not a very effective method of depositing particles on the oral surfaces and may be rather awkward for the user. Accordingly a deposition device may be employed, such as one which employs a spray jet (water and air) to deposit the two different particle suspensions. An exemplary deposition device may thus include two separate fluid suspension reservoirs, fluid pumps and two separate nozzles which both provide a jet in the same location. The device is able to deliver subsequent multiple shots, and is able to alternate the shots between the two suspension systems, starting with the positively charged particle system.

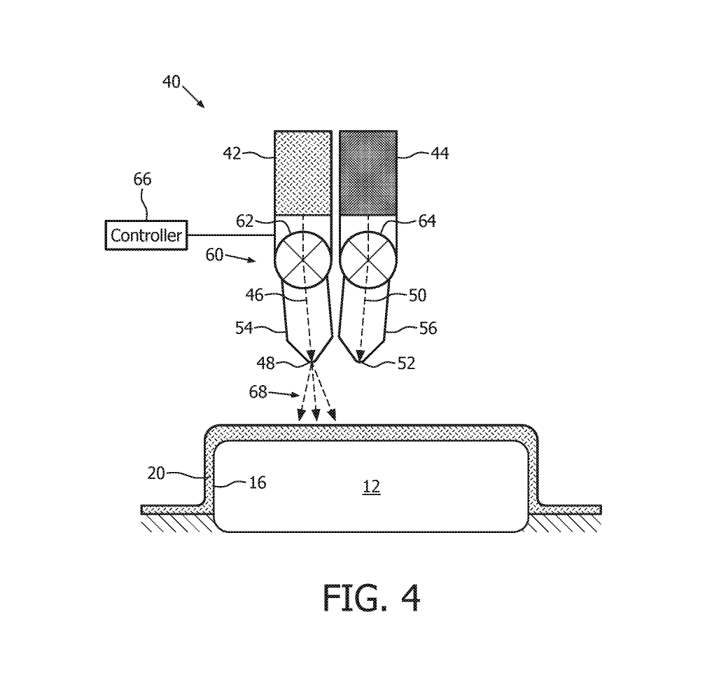

With reference now to FIGS. 4-6, the build-up of layers is illustrated graphically. The particles can be delivered by a deposition device 40, as shown, however, the method of application or type of deposition device is not limited. The deposition device includes a first reservoir 42 which holds or receives a supply of the positively-charged particles 36. A second reservoir 44 holds or receives a supply of the negatively-charged particles 38. The reservoirs 42, 44 may be charged from multi-dose containers of particles or from individual canisters containing an amount suitable for one application (not shown).

A first fluid pathway 46 connects the first reservoir with an outlet 48, from which the particles 36 are delivered to the surface to be coated. A second fluid pathway 50 connects the second reservoir 44 with an outlet 52 (which can be the same or different from outlet 48), from which the particles 38 are delivered to the surface to be coated. In the illustrated embodiment, the first and second pathways are defined by respective nozzles 54, 56 which each have a respective outlet 48, 52, although it is to be appreciated that these could feed to a common outlet. The outlet(s) 48, 52 may be shaped to apply an even layer of particles in the desired location.

The particles may be delivered in any suitable fluid, such as a compressed gas, e.g. air, a pressurized liquid, or a mixture thereof. The device includes a delivery mechanism 60 which alternately delivers first one then the other of the types of particles, e.g., in one or more short bursts sufficient to apply a monolayer of particles, to the surface 16, 18. For example, the delivery mechanism 60 may include a first delivery mechanism, such as a first pump 62 which delivers a short burst (or more than one) of particles 36 to nozzle 54 and a second delivery mechanism, such as a second pump 64, which delivers a short burst (or more than one) of particles 38 to nozzle 56. The pumps 62, 64 may be under the control of an electronic controller 66 (comprising memory and a processing device) which selectively actuates the pumps 62, 64. In another embodiment, valves connecting the respective reservoirs 42, 44 are selectively opened, e.g., by the controller. This allows the particles to flow to the nozzles under the force of a fluid, such as pressurized air and/or water connected with the respective reservoir.

In each case, when the first delivery mechanism 62 is actuated so as to deliver the positively-charged particles 36, the particles 36 are delivered from reservoir 42 into the first fluid flow path 46 and out of the outlet 48, in a fine stream of particles, which, because of their positive charge, readily adhere to the teeth or gums, forming a monolayer 20. In this step (S102), the second delivery mechanism 64 is prevented from delivering the second particles 38.

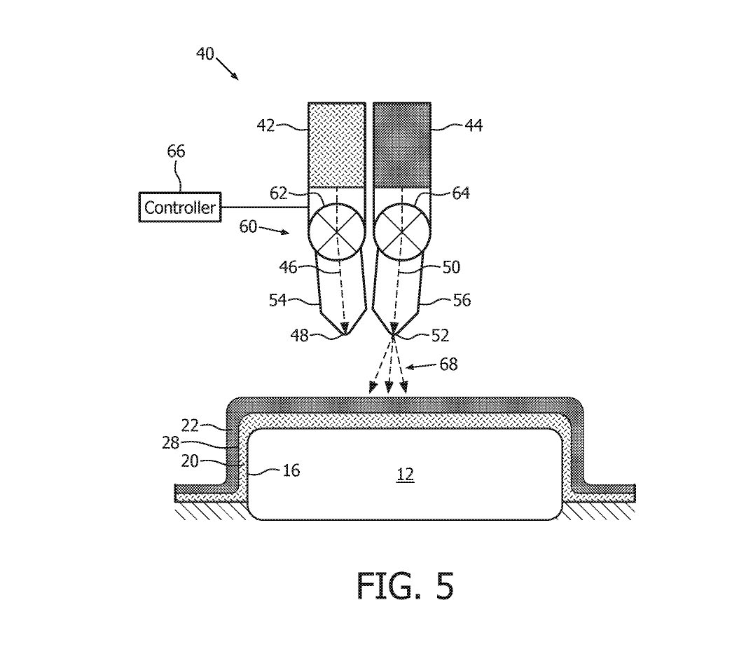

Similarly, as shown in FIG. 5, when the second delivery mechanism 64 is actuated so as to deliver the negatively-charged particles 38, a short burst of the particles 28 is delivered from reservoir 44 into the second fluid flow path 50 and out of the outlet 52, in a fine stream of particles, which, because of their negative charge, readily adhere to the particles 36 in the first (or preceding) positively-charged layer, forming a monolayer 22 on an outer surface 28 of the preceding layer 20. In this step (S104), the first delivery mechanism 62 is prevented from delivering the first particles 36.

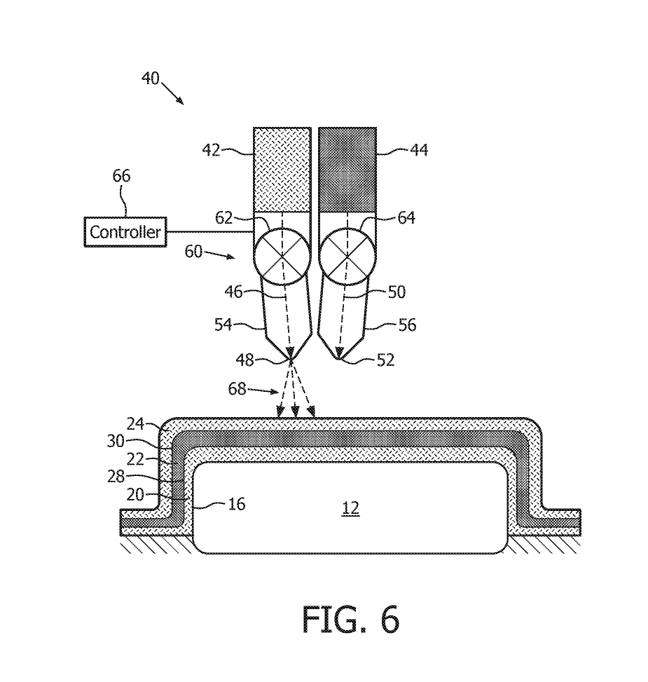

As shown in FIG. 6, the deposition of the third layer can proceed in the same manner as the first layer, with the third layer 24 of positively charged particles 36 being deposited on an outer surface 30 of the preceding (second) layer.

As will be appreciated, the deposition device 40 is not limited to two types of particles 36, 38, but could be adapted to include three or more reservoirs and delivery systems, etc. for delivering three or more types of particles, at least one type being positively charged and at least one type being negatively charged. In another embodiment, the delivery system employs only a single pump instead of two. For example, the pump may pump from the two different particle suspensions at the same time. In this way, if the subsequent delivery to the teeth is immediate the particles do not have time to agglomerate before reaching the teeth, so they still can form layers on the teeth. As will be appreciated, the release mechanism 60 may be upstream of the reservoir.

For example a deposition device 40 similar to the Philips Sonicare AirFloss™ device can be used for delivering the particles. Using suitable AirFloss spring force settings, water jet settings, nozzle configuration, and the like, adhesive sustained release gel particles can be efficiently delivered onto the tooth surface In one embodiment, the deposition device 40 is configured to allow the user to push a button when the device is properly positioned, upon which the device may then first generate a high speed shot of fluid (alone) (e.g., 20 to 30 m/s) for cleaning purposes (or, if desired multiple pulsed shots), and then alternate low speed shots (e.g. 0.5 m/s to 5 m/s, such as 1 m/s to 2 m/s), with particles, to deposit the layers.

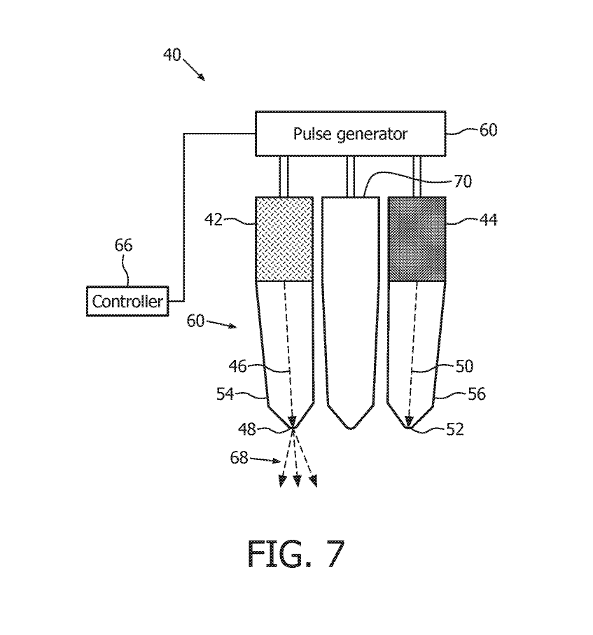

The deposition device may be a dedicated device, intended to be used after oral cleaning to deposit the sustained release system, as illustrated in FIGS. 4-6. Alternatively, the deposition device may be incorporated into an oral hygiene device for cleaning the teeth and/or gums, as illustrated, for example, in FIG. 7. In this embodiment, the deposition device 40 may include a cleaning nozzle 70 for delivering a cleaning fluid (e.g., air and/or water) and two additional nozzles 54, 56, aimed at the same location for deposition. After the cleaning shot(s), the device 40 fires a number of additional shots from the two side nozzles 54, 56 alternating the fluid pumps of the positively-charged and the negatively-charged particles. The device may utilize three separate fluid delivery systems, for the cleaning and the two particle suspensions, but in another embodiment, may use a common air pulse generator 60 as the delivery mechanism, to propel all three liquids to the teeth in the correct order. Separate liquids can be provided for cleaning (e.g., plain water) and for particle delivery (e.g., liquid containing one or more oral care agents, or a source of oral care particles that can be in situ combined with jetted water).

The first particles 36 have strong adhesive properties because of their positive net surface charge, electrostatically binding to the negatively charged pellicle on the teeth. Since the positively charged particles tend to repel each other, only maximally a monolayer of such particles can generally be deposited. By using subsequent shots of positively-charged particles 36 (e.g., chitosan-based) and negatively-charged particles 38 (e.g., alginate-based), much thicker particle multi-layers can be deposited on the target surface. Such thicker layers are beneficial for sustaining the beneficial effect of the therapeutic agents for a much longer time.

It is desirable to keep the positively and negatively-charged particles separated, prior to deposition in the oral cavity, in order to prevent them from agglomerating. This aids in keeping the particles individually in suspension.

Accordingly, providing separate reservoirs of the positively and negatively-charged particles or otherwise providing separate sources of the particles is desirable. The positively-charged particles in the first reservoir 42 repel each other and the negatively-charged particles in the second reservoir 44 repel each other. In the exemplary embodiment, therefore, the first reservoir 42 thus contains only positively charged particles and no negatively charged particles, and the second reservoir 44 contains only negatively-charged particles and no positively-charged particles. However, a small amount of positively-charged particles, such as less than 5 wt %, in the reservoir 44, and vice versa, may not unduly influence performance.

In the exemplary embodiment, different suspensions are formulated, one with positively-charged particles 36 and the other with negatively-charged particles 38.

### Forming Hydrogel Particles

Any method for forming hydrogel particles can be employed that is suitable to its respective components.

Following formation, fragmentation may be used to reduce the particle size. In the fragmentation process the polymeric material is broken into smaller pieces that retain the material properties of the parent material. This may be accomplished by a number of methods, including syringe to syringe mixing of flowable polymer materials, maceration of the polymer material with blades, rotors, hammers, ultrasonic vibrations, or other suitable techniques, filing, sanding, grating, and grinding and/or milling processes, such as cone and gyratory crushing, disk attrition milling, colloid and roll milling, screen milling and granulation, hammer and cage milling, pin and universal milling, jet or fluid energy milling, impact milling and breaking, jaw crushing, roll crushing, disc milling, and vertical rolling, including cryogenic grinding and/or cryogenic milling.

As an example, chitosan gel particles are manufactured by adding chitosan (highly viscous) powder to an acidic aqueous solution and the chitosan allowed to dissolve. Subsequently, the chitosan solution is introduced, e.g., dripped or sprayed, into an alkaline aqueous solution, which causes a gel to form. The chitosan gel (e.g., in the form of balls) may then be fragmented to form smaller particles, e.g., in the range of 20 to 200 micrometers. The pH of the suspension may be lowered to close to neutral, e.g., with a buffer. The particles may be used in the suspension. Alternatively, the particles may be removed from the suspension and washed with an aqueous washing solution.

The oral care agent may be introduced prior to gelation, e.g., combined with the chitosan powder or introduced to the aqueous acidic or alkaline solution, or a combination thereof. As an example, cetylpyridinium chloride could be present at from 0.1 to 5 g/liter, e.g., about 1 g/liter of the aqueous acidic or alkaline solution.

There may be no need to fragment the particles if they are sufficiently small, e.g., when produced by spraying.

Similarly, for manufacturing negatively-charged particles, a water-soluble alginate salt may be added to water and left to dissolve. The alginate solution is introduced, e.g., dripped or sprayed into a solution containing a metal salt (e.g., CaCl₂ solution) which forms an insoluble alginate salt. The drops quickly transform to gel particles when mixed with calcium chloride. The oral care agent may be introduced prior to gelation, e.g., combined with the water-soluble alginate salt, introduced to the alginate solution, or the metal salt solution, or a combination thereof.

While the method, delivery system, and deposition device have been described in terms of delivery of oral care agents to an oral cavity, it is also contemplated that they may be employed for generating a coating for delivery of therapeutic agents for other applications, such as to the skin or to surfaces within a person's body body.

Without intending to limit the scope of the exemplary embodiment, the following examples illustrate forming the delivery system 10.

### EXAMPLES

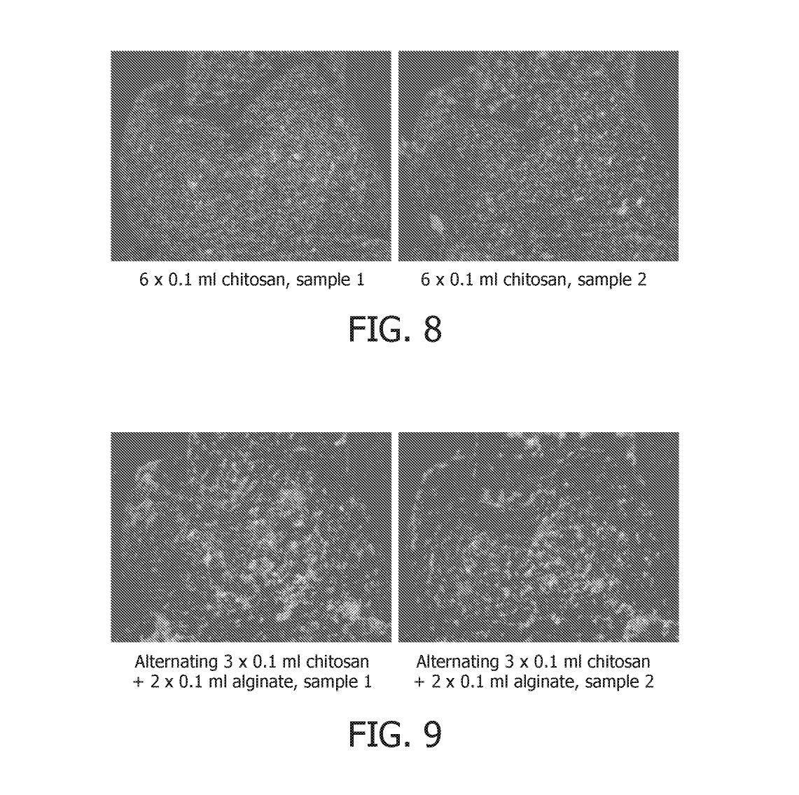

Black, tooth-shaped models were formed of polyamide (PA11), which has similar physical surface properties to human teeth in a typical oral cavity and were therefore used as model surfaces for evaluating deposition on tooth surfaces, as deposition of clear or light colored particles would photograph well against a black background. FIG. 8 shows macroscopic photographic images of the control group. In the control group, multiple deposition layers of positively charged gel particles (chitosan gel) are applied to the simulated tooth surface. The later depositions do not appear to add to more particles, and the surface seems to be saturated with particles rather quickly: new deposition shots do not lead to more volume deposited. This is expected, since the positively charged particles on the surface repel newly incoming positively charged particles. This was confirmed using 3D scans, where in the control group the adhering particles are maximally 0.1 mm thick, suggesting single layer particle deposition on the surface.

A different behavior is seen on the test group, as shown in FIG. 9, where alternating layers of positively and negatively charged gel particles were applied to the simulated tooth surface. With every positive layer deposition (chitosan gel), the particle agglomeration grows thicker. Since the negatively charged particles (alginate gel) are transparent they do not appear in the photographs, but since the white positively charged deposits grew much thicker with the negative layers in between, it can be concluded that multilayer structures of positively and negatively charged particles are built. This was confirmed using 3D scans, where in the test group, the adhering particles are up to 0.5 mm thick, suggesting single layer particle deposition on the surface. From this it can be concluded that depositing negatively charged particles in between deposited positively charged particles results in multiple layer deposition, capable of delivering a much larger volume of sustained release material on the teeth than is possible with positively charged particles alone.

Except where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. It is to be understood that the upper and lower amount, range, and ratio limits set forth herein may be independently combined. Similarly, the ranges and amounts for each element of the invention may be used together with ranges or amounts for any of the other elements. As used herein any member of a genus (or list) may be excluded from the claims.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A deposition method comprising:
depositing positively-charged particles on a surface of an oral cavity;
depositing at least one of negatively-charged particles and negatively-charged molecules on the positively-charged particles, an oral care agent being incorporated in at least one of the positively-charged particles and the negatively-charged particles, where present; and
optionally, repeating the depositing of the positively-charged particles on the at least one of negatively-charged particles and negatively-charged molecules.

2. The method of claim 1, wherein at least one of the positively-charged particles and negatively-charged particles comprises hydrogel particles.

3. The method of claim 1, wherein the positively-charged particles are selected from polysaccharides, polylysine, and polyacrylates, optionally functionalized with at least one of amine and quaternary ammonium groups.

4. The method of claim 1, wherein the positively-charged particles comprise a polysaccharide or a derivative thereof.

5. The method of claim 4, wherein the positively-charged particles comprise chitosan, or a derivative thereof.

6. The method of claim 1, wherein the negatively-charged particles are selected from alginate, carrageenan, and polymers functionalized with at least one of acid, sulfate and phosphate groups.

7. The method of claim 1, wherein a first layer of positively-charged particles is formed by said depositing of positively-charged particles, and a second layer of negatively-charged particles is formed by said depositing of negatively-charged particles.

8. The method of claim 7, wherein the positively-charged particles include a first active agent and the negatively-charged particles include a second active agent, different from the first active agent.

9. The method of claim 1, wherein at least one of the positively-charged particles and the negatively-charged particles are at least 10 μm in size.

10. The method of claim 1, wherein at least one of the positively-charged particles and the negatively-charged particles are up to up to 0.2 mm in size.

11. The method of claim 1, wherein a thickness (t) of the deposited particles, or particles and molecules, is at least 0.1 mm.

12. The method of claim 1, wherein a thickness (t) of the deposited particles, or particles and molecules, is up to 5 mm.

13. The method of claim 7, further comprising depositing at least a third layer in contact with one of the first and second layers, the third layer being configured as for the other of the first and second layers.

14. (canceled)

15. A deposition device comprising:
a first reservoir which holds positively-charged particles;
a second reservoir which holds negatively-charged particles, at least one of the positively-charged particles and negatively-charged particles incorporating an active agent; and
a delivery mechanism which delivers the positively charged particles and the negatively-charged particles to an associated surface.
